(19) United States
(12) Patent Application Publication
(10) Pub. No.: US 2025/0244197 A1
(43) Pub. Date: Jul. 31, 2025

(54) DETECTING PACKAGED PRODUCTS WITH IMPROPER VACUUM SEALS

(71) Applicant: Marble, Inc., Cambridge, MA (US)

(72) Inventors: Geethika Weliwitigoda, Westford, MA (US); Tyler Randolph, Edgefield, SC (US)

(21) Appl. No.: 19/038,128

(22) Filed: Jan. 27, 2025

Related U.S. Application Data
(60) Provisional application No. 63/625,535, filed on Jan. 26, 2024.

(51) Int. Cl. G01M 3/38; G01N 21/88; G01N 33/00; G01N 35/04

(52) U.S. Cl. CPC G01M 3/38; G01N 21/8806; G01N 21/8851; G01N 33/0081; G01N 35/04

(57) ABSTRACT

This disclosure includes techniques for evaluating a vacuum seal in a packaged product. A device controls a lighting system to direct light at a packaged product. The device controls a camera system to capture one or more images of the packaged product while the lighting system is directing the light at the packaged product. The device receives the one or more images of the packaged product captured while the lighting system is directing light at the packaged product. The device analyzes one or more characteristics of the light in the one or more images. The device determines, based on the one or more characteristics of the light in the one or more images, a quality score indicating whether the packaged product was properly vacuum sealed.

### RELATED APPLICATIONS

This application claims the benefit of U.S. Provisional Application No. 63/625,535, filed Jan. 26, 2024, the entire contents of which are incorporated herein by reference.

### TECHNICAL FIELD

The disclosure relates to vacuum seal evaluation.

### BACKGROUND OF THE INVENTION

In meat processing facilities, livestock carcasses are broken down into smaller cuts of meat, known as subprimals (e.g., brisket, chuck roll, etc.). The subprimals are manually placed into bags and sent through vacuum seal chambers. Meat products are vacuum sealed in plastic bags to remove oxygen and prevent contaminants from reaching the meat. Such packaging reduces the growth of bacteria and prevents discoloration, a critical aspect of food quality.

Not all plastic bags are successfully vacuum-sealed. These defective packages are colloquially referred to as “leakers,” as the packages may leak liquid and make a mess if undetected. Meat processing facilities report a leaker rate between 2-5%. Other failures could include where the seal is intact, but extraneous air is included on an interior portion of the package.

The vacuum-sealed packages of meat are discharged from the vacuum sealer onto a conveyor that carries the meat packages to the boxing area. Operators are tasked with checking for leaker products prior to boxing. If a leaker is identified, it is sent upstream for repackaging, creating waste in packaging material and energy use.

Currently, meat processing facilities detect leakers manually. Human operators visually inspect packages for the presence of air bubbles, loose plastic, plastic wrinkles, or abnormalities in the seal line. In some cases, the operator must pick up the product, either to perform a closer visual inspection or to “feel” the looseness of the plastic bag around the product.

Trace gas detection is used inline in some applications that use modified atmosphere packaging. This may mainly be used with food tray packaging applications and are not aware of any meat processing facilities using this technology with vacuum-sealed bags.

Many offline methods of leaker detection are available, often involving water tanks. These methods cannot be applied to a production line to test every package at the speed (e.g., 40 pieces per minute) required by meat processing facilities.

### SUMMARY OF THE INVENTION

In general, the disclosure is directed to one or more techniques for evaluating a vacuum seal in a packaged product. A device controls a lighting system to direct light at a packaged product. The device controls a camera system to capture one or more images of the packaged product while the lighting system is directing the light at the packaged product. The device receives the one or more images of the packaged product captured while the lighting system is directing light at the packaged product. The device analyzes one or more characteristics of the light in the one or more images. The device determines, based on the one or more characteristics of the light in the one or more images, a quality score indicating whether the packaged product was properly vacuum sealed.

A leaker detection system may be a component of an automated pack-off system which aims to reduce the labor required to box meat products by 50% or more. Today, operators spot the most obvious leakers (>50%) as the leaker product passes by on the conveyor belt. These are routed to the repackaging area with no effort from the operator. They simply let the package pass by. If an automated pack-off system sorts these leaker products into its chutes, the operator will now have to pick up every leaker and place it back on the conveyor belt. Such added effort jeopardizes the labor efficiencies created by the automatic sorting and more ergonomic box packing of the pack-off system. An automated leaker detection system as described herein may alert the supervisory software of the automated pack-off that the leaker product should not be sorted and should instead be routed for repackaging.

Adding leaker detection to the pack-off system may also improve integration with robotic boxing. With robotic boxing, the human operator is no longer in place to perform manual inspection/quality assurance.

Subtle leakers are sometimes missed by busy human operators, especially those with less training and experience, and shipped to customers. A highly accurate leaker detection system, such as that described herein, can improve quality assurance and reduce food waste.

The current manual system makes it difficult to measure the defect rate and isolate the cause of defects. As such, intervention to reduce leaker frequency is difficult and unlikely. Using an automated system creates an opportunity to apply data analysis to identify the cause of leakers and reduce the frequency of defective packages.

A byproduct of leaker creation is plastic packaging waste as unsealed bags are cut open and discarded so the meat product can be repackaged and resealed in a new bag. It is estimated that 31 million pieces of beef may be rebagged each year with 31 million bags being discarded. Greater visibility into the frequency and pattern of leaker defects (e.g., increased frequency on a specific line or for specific products or operators) could allow more frequent and targeted interventions to reduce packaging material and energy waste.

In one example, the disclosure is directed to a method in which one or more processors control a lighting system to direct light at a packaged product. The method further includes controlling, by the one or more processors, a camera system to capture one or more images of the packaged product while the lighting system is directing the light at the packaged product. The method also includes receiving, by the one or more processors, the one or more images of the packaged product captured while the lighting system is directing light at the packaged product. The method further includes analyzing, by the one or more processors, one or more characteristics of the light in the one or more images. The method also includes determining, by the one or more processors and based on the one or more characteristics of the light in the one or more images, a quality score indicating whether the packaged product was properly vacuum sealed.

In another example, the disclosure is directed to a packing system comprising a lighting system, a camera system, and one or more processors. The one or more processors are configured to control the lighting system to direct light at a packaged product. The one or more processors are further configured to control the camera system to capture one or more images of the packaged product while the lighting system is directing the light at the packaged product. The one or more processors are also configured to receive the one or more images of the packaged product captured while the lighting system is directing light at the packaged product. The one or more processors are further configured to analyze one or more characteristics of the light in the one or more images. The one or more processors are also configured to determine, based on the one or more characteristics of the light in the one or more images, a quality score indicating whether the packaged product was properly vacuum sealed.

In another example, the disclosure is directed to a non-transitory computer-readable storage medium containing instructions. The instructions, when executed, cause one or more processors to control a lighting system to direct light at a packaged product. The instructions, when executed, further cause one or more processors to control a camera system to capture one or more images of the packaged product while the lighting system is directing the light at the packaged product;

The instructions, when executed, also cause one or more processors to receive the one or more images of the packaged product captured while the lighting system is directing light at the packaged product. The instructions, when executed, further cause one or more processors to analyze one or more characteristics of the light in the one or more images. The instructions, when executed, also cause one or more processors to determine, based on the one or more characteristics of the light in the one or more images, a quality score indicating whether the packaged product was properly vacuum sealed.
In Example 1, a method comprises controlling, by one or more processors, a lighting system to direct light at a packaged product; controlling, by the one or more processors, a camera system to capture one or more images of the packaged product while the lighting system is directing the light at the packaged product; receiving, by the one or more processors, the one or more images of the packaged product captured while the lighting system is directing light at the packaged product; analyzing, by the one or more processors, one or more characteristics of the light in the one or more images; and determining, by the one or more processors and based on the one or more characteristics of the light in the one or more images, a quality score indicating whether the packaged product was properly vacuum sealed.
Example 2 relates to the method according to Example 1, wherein analyzing the one or more characteristics of the light comprises one or more of: analyzing one or more glare patterns of the light reflecting off the packaged product, and analyzing one or more scatter signatures of the light on the packaged product.
Example 3 relates to the method according to Example 2, wherein the one or more scatter signatures comprise manifestations of green wavelength spectrum light shined into the packaged product.
Example 4 relates to the method according to Example 3, wherein the lighting system comprises one or more lasers that emit green wavelength spectrum light.
Example 5 relates to the method according to any one or more of Examples 1-4, wherein the packaged product comprises a vacuum sealed food product.
Example 6 relates to the method according to any one or more of Examples 1-5, wherein the camera system comprises a plurality of camera devices.
Example 7 relates to the method according to Example 6, wherein the camera system further comprises a camera enclosure surrounding each respective camera of the plurality of cameras.
Example 8 relates to the method according to any one or more of Examples 6-7, wherein a first camera of the plurality of cameras is positioned above a conveyor carrying the packaged product, wherein a second camera of the plurality of cameras is positioned on a first side of the conveyor, and wherein a third camera of the plurality of cameras is positioned on a second side of the conveyor.
Example 9 relates to the method according to any one or more of Examples 1-8, wherein the packaged product comprises a first packaged product in a plurality of packaged products being carried by a conveyor, and wherein the method further comprises: controlling, by the one or more processors, a set of gapping conveyors to move the plurality of packaged products into a single row prior to passing the lighting system and the camera system.
Example 10 relates to the method according to any one or more of Examples 1-9, wherein analyzing the one or more characteristics of the light in the one or more images comprises: for each of the one or more images: inputting, by the one or more processors, the respective image into a model trained with previous images of packaged products that contain leaks and with previous images of packaged products that do not contain leaks; and comparing, by the one or more processors and using the model, the one or more characteristics of the light in the respective image to one or more characteristics of the light for the model; and determining, by the one or more processors, the quality score based on each of the comparisons for each of the one or more images.
Example 11 relates to the method according to any one or more of Examples 1-10, wherein each of the one or more images comprise one or more of: an image captured by a same camera device at a unique time to show a different portion of the packaged product, and an image captured by a different camera device to show a different angle of the packaged product.
Example 12 relates to the method according to any one or more of Examples 1-11, further comprising: in response to determining that quality score is below a seal score threshold, performing, by the one or more processors, a corrective action.
Example 13 relates to the method according to Example 12, wherein performing the corrective action comprises one or more of: outputting, by the one or more processors, an alert notifying a user of the leak in the packaged product, and controlling, by the one or more processors, a sorting mechanism to remove the packaged product from a conveyor carrying the packaged product.
Example 14 relates to the method according to any one or more of Examples 1-13, further comprising: in response to determining that the quality score is above a seal score threshold, controlling, by the one or more processors, a sorting mechanism to keep the packaged product on a conveyor carrying the packaged product.
Example 15 relates to the method according to any one or more of Examples 1-14, further comprising: in response to determining that the quality score is above a first seal score threshold but below a second seal score threshold, outputting, by the one or more processors, an alert for a user to manually inspect the packaged product.
Example 16 relates to the method according to any one or more of Examples 1-15, wherein the one or more characteristics of the light comprise one or more of: a glare pattern created by one or more of air bubbles, plastic wrinkles in the packaged product, haze on a plastic exterior of the packaged product, a color contrast between packaging of the packaged product and a product inside the packaging, blood or other liquid in or around a seal of the packaged product, wrinkles around the seal of the packaged product, contamination on one or both sides of the seal of the packaged product, a burn through of the seal of the packaged product, and a sign of the seal of the packaged product lacking integrity.
Example 17 relates to the method according to any one or more of Examples 1-16, wherein the lighting system comprises one or more of LED lights or fluorescent lights.
Example 18 relates to the method according to any one or more of Examples 1-17, wherein the quality score comprises one or more of: a probability of the packaged product being properly vacuum sealed, a probability of the packaged product being improperly vacuum sealed, and a quantitative value based on a comparison of the one or more images to one or more images of a machine learning model.
Example 19 relates to the method according to any one or more Examples 1-18, wherein the packaged product comprises a first packaged product of a plurality of packaged products, and wherein the method further comprises: for each of the plurality of packaged products: receiving, by the one or more processors, the one or more images of the respective packaged product captured while the lighting system is directing light at the respective packaged product; analyzing, by the one or more processors, one or more characteristics of the light in the one or more images of the respective packaged product; and determining, by the one or more processors and based on the one or more characteristics of the light in the one or more images of the respective packaged product, a quality score for the respective packaged product indicating whether the respective packaged product was properly vacuum sealed.
Example 20 relates to the method according to Example 19, further comprising: determining, by the one or more processors and based on each of the quality scores for the packaged products of the plurality of packaged products, trend data for the plurality of packaged products.
Example 21 relates to the method according to any one or more of Examples 19-20, further comprising: determining, by the one or more processors and based on the quality scores for the plurality of packaged products, a failure rate for the plurality of packaged products; comparing, by the one or more processors, the failure rate to a historical failure rate; in response to the failure rate exceeding the historical failure rate by a threshold amount, determining, by the one or more processors, that a production error is present.
Example 22 relates to the method according to Example 21, wherein the production error comprises one or more of a mechanical error, a user error, or a package quality error.
Example 23 relates to the method according to any one or more of Examples 21 or 22, further comprising: outputting, by the one or more processors, an indication of the production error.
Example 24 relates to the method according to any one or more of Examples 1-23, further comprising: estimating, by the one or more processors and based on the one or more images, an amount of air inside packaging of the packaged product; and determining, by the one or more processors, the quality score for the packaged product based at least in part on the one or more characteristics of the light and the estimated amount of air.
In Example 25, a packing system comprising: a lighting system; a camera system; and one or more processors configured to: control the lighting system to direct light at a packaged product; control the camera system to capture one or more images of the packaged product while the lighting system is directing the light at the packaged product; receive the one or more images of the packaged product captured while the lighting system is directing light at the packaged product; analyze one or more characteristics of the light in the one or more images; and determine, based on the one or more characteristics of the light in the one or more images, a quality score indicating whether the packaged product was properly vacuum sealed.
Example 26 relates to the packing system according to Example 25, wherein the one or more processors are further configured to perform the methods of any one or more of Examples 1-24.
Example 27 relates to the packing system according to Examples 25-26, wherein the system further comprises a conveyor that moves the packaged product throughout the packing system.
Example 28 relates to the packing system according to Example 27, further comprising a first angled panel on a first side of the conveyor and a second angled panel on a second side of the conveyor, wherein each of the first angled panel and the second angled panel are a same color as the conveyor such that the one or more images include the packaged product, the light directed at the packaged product, and a monotone background.
In Example 29, a method comprises performing any of the techniques of any combination of Examples 1-24 or using the system of Examples 25-28.
In Example 30, a device is configured to perform any of the methods of any combination of Examples 1-24 or using the system of Examples 25-28.
In Example 31, an apparatus comprises means for performing any of the method of any combination of Examples 1-24 or using the system of Examples 25-28.
In Example 32, a non-transitory computer-readable storage medium has stored thereon instructions that, when executed, cause one or more processors of a computing device to perform the method of any combination of Examples 1-24 or using the system of Examples 25-28.
In Example 33, a system comprises one or more computing devices configured to perform a method of any combination of Examples 1-24.
In Example 34, any of the techniques described herein are performed.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present disclosure and therefore do not limit the scope of the invention. The drawings are not necessarily to scale, though examples can include the scale illustrated, and are intended for use in conjunction with the explanations in the following detailed description wherein like reference characters denote like elements. Examples of the present disclosure will hereinafter be described in conjunction with the appended drawings.

FIG. 1 is a perspective view of a product processing and packing system receiving products from a vacuum-sealing system, in accordance with the techniques of this disclosure.

FIG. 2 is a block diagram illustrating a more detailed example of a computing device configured to perform the techniques described herein.

FIGS. 3A-3B are different perspective views of a lighting system and a camera system that are integrated into a product processing and packing system, in accordance with one or more techniques of this disclosure.

FIG. 4 is a perspective view of a lighting system and a camera system that are integrated into a product processing and packing system, in accordance with to one or more techniques of this disclosure.

FIGS. 5A-5G are different perspective views of a lighting system and a camera system that are integrated into a product processing and packing system, in accordance with one or more techniques of this disclosure.

FIGS. 6A-6G are different perspective views of a product processing and packing system that includes a lighting system and a camera system, in accordance with one or more techniques of this disclosure.

FIG. 7 is an example set of images of packaged products without detected leaks, in accordance with one or more techniques of this disclosure.

FIG. 8 is an example set of images of packaged products with detected leaks, in accordance with one or more techniques of this disclosure.

FIG. 9 is a flow diagram illustrating an example method for evaluating a vacuum seal on a packaged product, in accordance with one or more techniques of this disclosure.

### DETAILED DESCRIPTION

The following detailed description is exemplary in nature and is not intended to limit the scope, applicability, or configuration of the techniques or systems described herein in any way. Rather, the following description provides some practical illustrations for implementing examples of the techniques or systems described herein. Those skilled in the art will recognize that many of the noted examples have a variety of suitable alternatives.

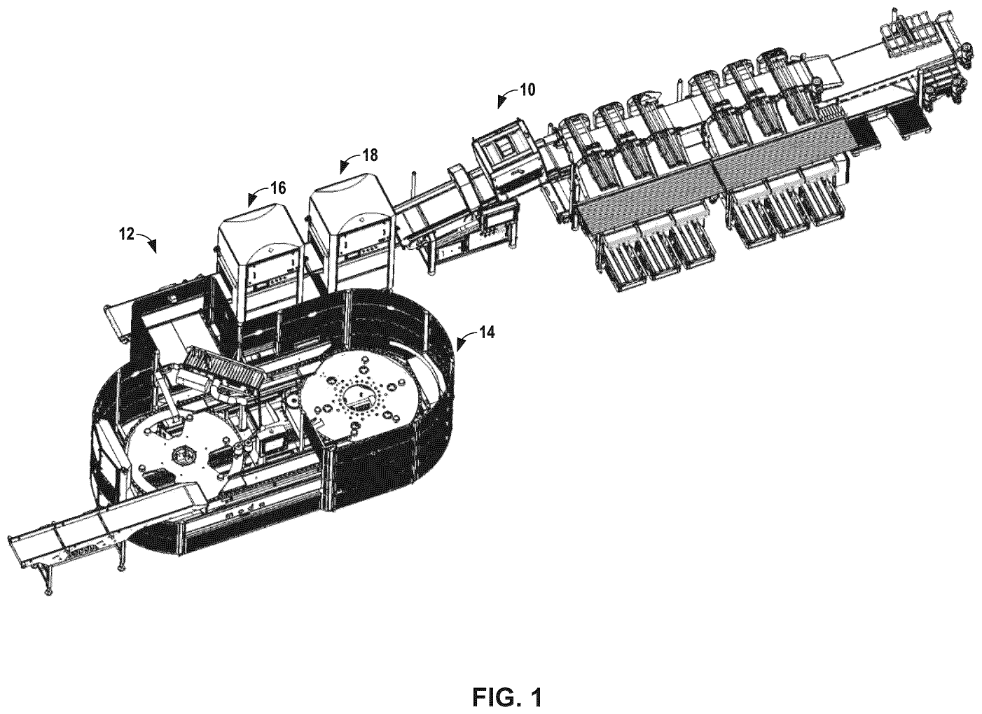

In certain implementations, the spacing conveyor and classification conveyor (also referred to as a classification system) can be used in conjunction with or incorporated into a product processing and packing system. One exemplary system embodiment 10 is shown in FIG. 1, in which the product processing and packing system 10 receives meat products from a known vacuum-sealing system 12 that vacuum seals each individual meat product. The vacuum-sealing system 12 has a rotary machine 14, a shrink tunnel 16 that that vacuums and seals the meat product into a packaging and uses hot water to shrink the bag tighter around the meat, and a blower 18 that dries the vacuum-sealed meat product. The processing system 10 receives the vacuum-sealed meat product from the blower 18 and classifies, sorts, and ultimately packs that product into a box or other bulk packaging. In other embodiments, the pack-off system 10 and any other system embodiment disclosed or contemplated herein receives meat products from any known vacuum-sealing system or any other product conveyance system. Further, the various pack-off systems herein can receive meat products which are packaged or unpackaged. In further alternatives, any of the exemplary pack-off systems herein may be configured to receive other types of products.

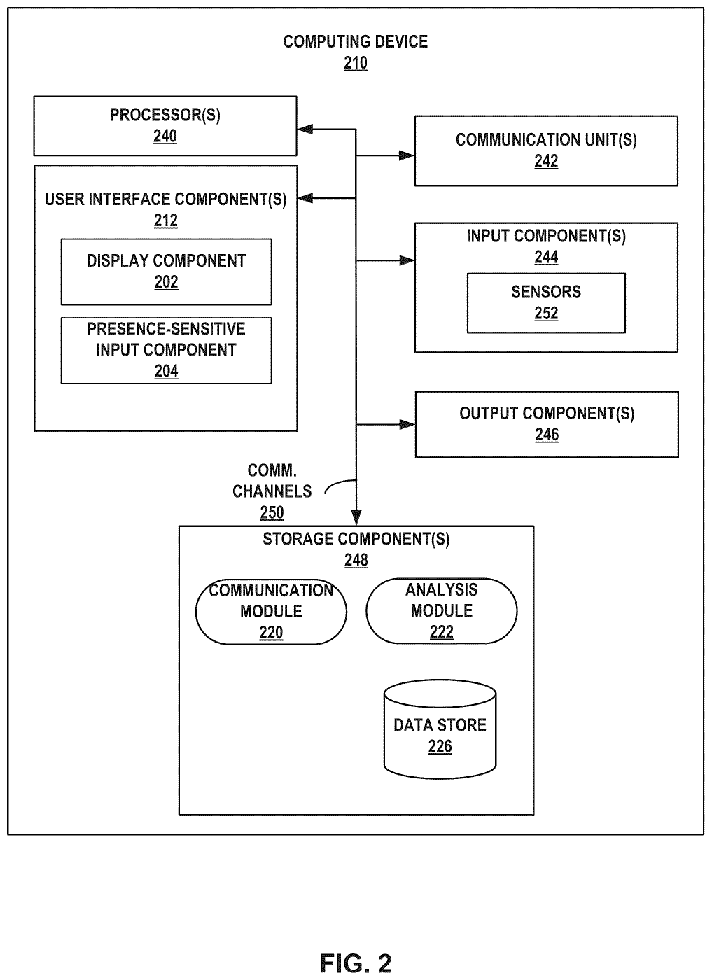

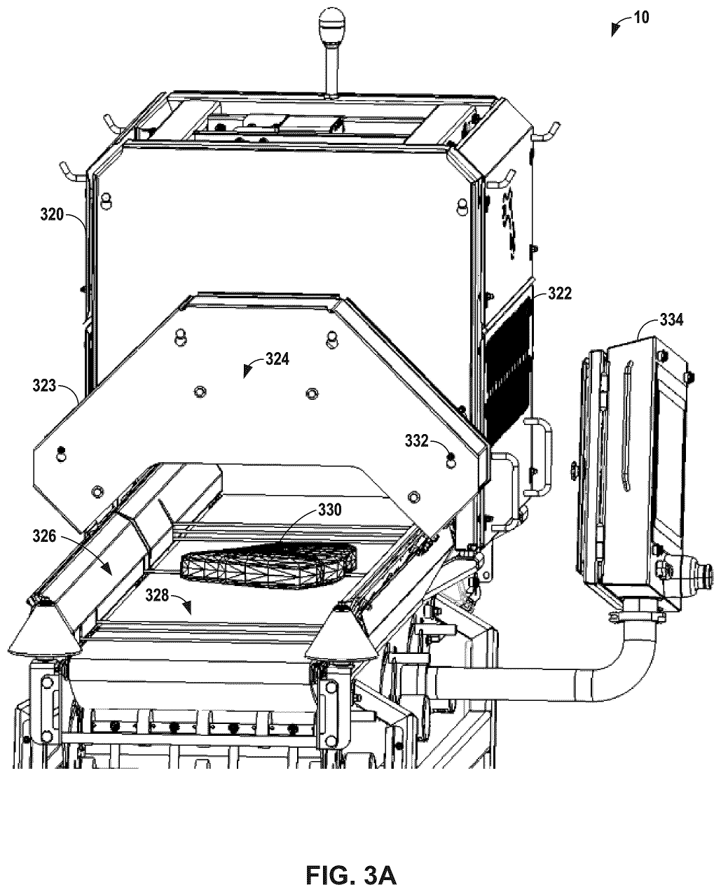

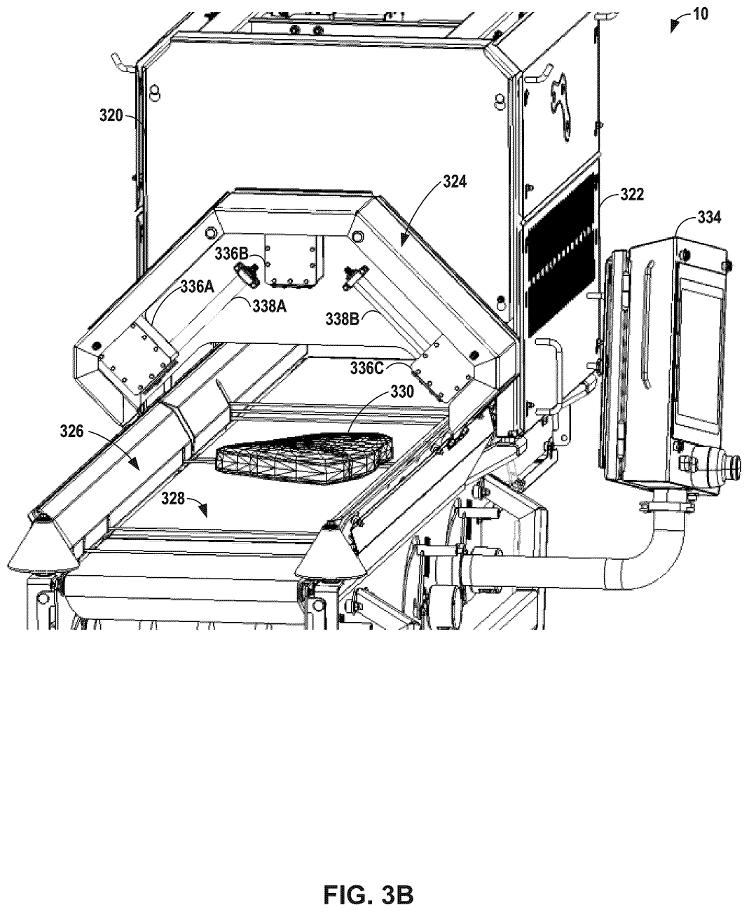

One embodiment of the seal evaluation system incorporated into the exemplary product processing system 10 of FIG. 1 is shown in additional detail in FIGS. 3A-6G. More specifically, the lighting and camera system shown in FIGS. 3A-6G may be placed between processing system 10 and vacuum-sealing system 12 such that the lighting and camera system (including a computing device, such as computing device 210 of FIG. 2) may be utilized to evaluate the quality of the vacuum seal of the packaged product and to determine if the packaged product was properly vacuum sealed prior to the packaged product being sorted and boxed for shipment.

A full description of the product processing and packing system embodiments into which any of the various spacing and/or classification devices can be incorporated is disclosed in U.S. patent application Ser. No. 18/449,537, entitled “Product Classification, Sorting, and Packing Systems and Methods,” which was filed on Aug. 14, 2023 and is hereby incorporated herein by reference in its entirety.

The techniques of this disclosure may include a computing device using of computer vision technology to detect defective vacuum-sealed packages in real-time in a production setting at production speeds. The system involves a number of cameras, including up to three or more cameras, and a lighting rig attached to the conveyor belt carrying the vacuum-sealed meat products. These techniques may employ multiple methodologies to make the visual features of leaker packages detectable within an RGB image. One such method may include the analysis of glare patterns caused by harsh lighting reflecting off the plastic of a bagged product (e.g., a glare-based method). A secondary or alternative method may be the analysis of the scatter signatures that manifest on the plastic when light sources of the green wavelength spectrum (lasers) are shined onto a leaked product. Other methodologies may include the analysis of other features of the package, such as seam analysis of packages or air detection within the package.

The system and techniques described herein integrate the classification of leaker or non-leaker for each product into the product routing decision of an automated pack-off system so that leaker products bypass the chutes and boxing stations and route to the leaker repackaging area of the facility. Some examples could have a two-conveyor belt with a line-scanning camera capturing the bottom side of the product.

FIG. 2 is a block diagram illustrating a detailed example of a computing device configured to perform the techniques described herein. FIG. 2 illustrates only one particular example of computing device 210, and many other examples of computing device 210 may be used in other instances and may include a subset of the components included in example computing device 210 or may include additional components not shown in FIG. 2.

Computing device 210 may be any computer with the processing power required to adequately execute the techniques described herein. For instance, computing device 210 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), a vehicle, a wearable computing device (e.g., a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein.

As shown in the example of FIG. 2, computing device 210 includes user interface components (UIC) 212, one or more processors 240, one or more communication units 242, one or more input components 244, one or more output components 246, and one or more storage components 248. UIC 212 includes display component 202 and presence-sensitive input component 204. Storage components 248 of computing device 210 include communication module 220, analysis module 222, and data store 226.

One or more processors 240 may implement functionality and/or execute instructions associated with computing device 210 to control an automated pack-off system and analyze images of packaged products to determine whether the packaged products were properly vacuum sealed. That is, processors 240 may implement functionality and/or execute instructions associated with computing device 210 determine whether the automated pack-off system is packing the packaged products properly.

Examples of processors 240 include any combination of application processors, display controllers, auxiliary processors, one or more sensor hubs, and any other hardware configured to function as a processor, a processing unit, or a processing device, including dedicated graphical processing units (GPUs). Modules 220 and 222 may be operable by processors 240 to perform various actions, operations, or functions of computing device 210. For example, processors 240 of computing device 210 may retrieve and execute instructions stored by storage components 248 that cause processors 240 to perform the operations described with respect to modules 220 and 222. The instructions, when executed by processors 240, may cause computing device 210 to control an automated pack-off system and analyze images of packaged products to determine whether the packages products were properly vacuum sealed.

Communication module 220 may execute locally (e.g., at processors 240) to provide functions associated with sending control signals to lighting systems and camera systems, as well as receiving data from either of these systems. In some examples, communication module 220 may act as an interface to a remote service accessible to computing device 210. For example, communication module 220 may be an interface or application programming interface (API) to a remote server that outputs the control signals to the lighting system and the camera system and receive data in return.

In some examples, analysis module 222 may execute locally (e.g., at processors 240) to provide functions associated with analyzing images received from a camera system and determining whether packaged products are properly vacuum sealed. In some examples, analysis module 222 may act as an interface to a remote service accessible to computing device 210. For example, analysis module 222 may be an interface or application programming interface (API) to analyze images received from a camera system and determine whether packaged products are properly vacuum sealed.

One or more storage components 248 within computing device 210 may store information for processing during operation of computing device 210 (e.g., computing device 210 may store data accessed by modules 220 and 222 during execution at computing device 210). In some examples, storage component 248 is a temporary memory, meaning that a primary purpose of storage component 248 is not long-term storage. Storage components 248 on computing device 210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 220 and 222 and data store 226. Storage components 248 may include a memory configured to store data or other information associated with modules 220 and 222 and data store 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of computing device 210 may communicate with external devices via one or more wired and/or wireless networks by transmitting and/or receiving network signals on one or more networks. Examples of communication units 242 include a network interface card (e.g., such as an Ethernet card), an optical transceiver, a radio frequency transceiver, a GPS receiver, a radio-frequency identification (RFID) transceiver, a near-field communication (NFC) transceiver, or any other type of device that can send and/or receive information. Other examples of communication units 242 may include short wave radios, cellular data radios, wireless network radios, as well as universal serial bus (USB) controllers.

One or more input components 244 of computing device 210 may receive input. Examples of input are tactile, audio, and video input. Input components 244 of computing device 210, in one example, include a presence-sensitive input device (e.g., a touch sensitive screen, a PSD), mouse, keyboard, voice responsive system, camera, microphone or any other type of device for detecting input from a human or machine. In some examples, input components 244 may include one or more sensor components (e.g., sensors 252). Sensors 252 may include one or more biometric sensors (e.g., fingerprint sensors, retina scanners, vocal input sensors/microphones, facial recognition sensors, cameras), one or more location sensors (e.g., GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a radar sensor, a lidar sensor, a sonar sensor, a heart rate sensor, magnetometer, glucose sensor, olfactory sensor, compass sensor, or a step counter sensor.

One or more output components 246 of computing device 210 may generate output in a selected modality. Examples of modalities may include a tactile notification, audible notification, visual notification, machine generated voice notification, or other modalities. Output components 246 of computing device 210, in one example, include a presence-sensitive display, a sound card, a video graphics adapter card, a speaker, a cathode ray tube (CRT) monitor, a liquid crystal display (LCD), a light emitting diode (LED) display, an organic LED (OLED) display, a virtual/augmented/extended reality (VR/AR/XR) system, a three-dimensional display, or any other type of device for generating output to a human or machine in a selected modality.

UIC 212 of computing device 210 may include display component 202 and presence-sensitive input component 204. Display component 202 may be a screen, such as any of the displays or systems described with respect to output components 246, at which information (e.g., a visual indication) is displayed by UIC 212 while presence-sensitive input component 204 may detect an object at and/or near display component 202.

While illustrated as an internal component of computing device 210, UIC 212 may also represent an external component that shares a data path with computing device 210 for transmitting and/or receiving input and output. For instance, in one example, UIC 212 represents a built-in component of computing device 210 located within and physically connected to the external packaging of computing device 210 (e.g., a screen on a mobile phone). In another example, UIC 212 represents an external component of computing device 210 located outside and physically separated from the packaging or housing of computing device 210 (e.g., a monitor, a projector, etc. that shares a wired and/or wireless data path with computing device 210).

UIC 212 of computing device 210 may detect two-dimensional and/or three-dimensional gestures as input from a user of computing device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with the techniques of this disclosure, communication module 220 may control a lighting system to direct light at a packaged product. In some instances, the lighting system comprises one or more of LED lights, fluorescent lights, or any other high-intensity area light that can shine over a packaged product on a conveyor. In some instances, the packaged product may be a vacuum sealed food product, such as a meat or cheese product.

Communication module 220 may control a camera system to capture one or more images of the packaged product while the lighting system is directing the light at the packaged product. In some instances, the camera system may include a plurality of camera devices. In some such instances, the camera system may further include a camera enclosure surrounding each respective camera of the plurality of cameras. For example, a first camera of the plurality of cameras may be positioned above a conveyor carrying the packaged product, a second camera of the plurality of cameras may be positioned on a first side of the conveyor, and a third camera of the plurality of cameras may be positioned on a second side of the conveyor.

In some instances, 11. The method of any one or more of claims 1-10, wherein each of the one or more images may be an image captured by a same camera device at a unique time to show a different portion of the packaged product or an image captured by a different camera device to show a different angle of the packaged product.

Communication module 220 may receive the one or more images of the packaged product captured while the lighting system is directing light at the packaged product.

Analysis module 222 may analyze one or more characteristics of the one or more images, such as one or more characteristics of the light in the one or more images. For instance, in analyzing the one or more characteristics of the light, analysis module 222 may analyze one or more glare patterns of the light reflecting off the packaged product or analysis module 222 may analyze one or more scatter signatures of the light on the packaged product. When the one or more characteristics include the one or more scatter signatures, the scatter signatures may be manifestations of green wavelength spectrum light shined into the packaged product. In such instances, the lighting system may include one or more lasers that emit green wavelength spectrum light.

In other instances, the one or more characteristics of the images and/or the one or more characteristics of the light may include one or more of a glare pattern created by one or more of air bubbles, plastic wrinkles in the packaged product, haze on a plastic exterior of the packaged product, a color contrast between packaging of the packaged product and a product inside the packaging, blood or other liquid in or around a seal of the packaged product, wrinkles around the seal of the packaged product, contamination on one or both sides of the seal of the packaged product, a burn through of the seal of the packaged product, and a sign of the seal of the packaged product lacking integrity.

In some instances, in analyzing the one or more characteristics of the one or more images or of the light in the one or more images, for each of the one or more images, analysis module 222 may input the respective image into a model trained with previous images of packaged products that contain leaks and with previous images of packaged products that do not contain leaks. Analysis module 222 may compare, using the model, the one or more characteristics of the light in the respective image to one or more characteristics of the light for the model. Analysis module 222 may determine the quality score based on each of the comparisons for each of the one or more images.

Analysis module 222 may determine, based on the one or more characteristics of the light in the one or more images, a quality score indicating whether the packaged product was properly vacuum sealed. In some instances, the quality score may be any one or more of a probability of the packaged product being properly vacuum sealed, a probability of the packaged product being improperly vacuum sealed, and a quantitative value based on a comparison of the one or more images to one or more images of a machine learning model.

In some instances, the techniques of this disclosure may be applied across a series of products. For instance, the packaged product may be a first packaged product in a plurality of packaged products being carried by a conveyor. In such instances, communication module 220 may control a set of gapping conveyors to move the plurality of packaged products into a single row prior to passing the lighting system and the camera system.

For each of the plurality of packaged products, communication module 220 may receive the one or more images of the respective packaged product captured while the lighting system is directing light at the respective packaged product. Analysis module 222 may analyze one or more characteristics of the images and/or one or more characteristics of the light in the one or more images of the respective packaged product. Analysis module 222 may determine, based on the one or more characteristics of the light in the one or more images of the respective packaged product, a quality score for the respective packaged product indicating whether the respective packaged product was properly vacuum sealed.

In some such instances, analysis module 222 may further determine, based on each of the quality scores for the packaged products of the plurality of packaged products, trend data for the plurality of packaged products. For instance, analysis module 222 may determine, based on the quality scores for the plurality of packaged products, a failure rate for the plurality of packaged products. Analysis module 222 may compare the failure rate to a historical failure rate. In response to the failure rate exceeding the historical failure rate by a threshold amount, analysis module 222 may determining, by the one or more processors, that a production error is present. The production error may be any one or more of a mechanical error (e.g., the vacuum sealer or some other portion of the automatic pack-off system is improperly handling the packaged products), a user error (e.g., users are improperly placing the products in the system or are attempting to run the system at too high of a capacity), or a package quality error (e.g., a same bag used for one or more products may come from a defective batch).

When analyzing the one or more images, analysis module 222 may be capable of determining what type of product is included in the one or more images. A full description of the product processing and packing system embodiments into which any of the product identification devices can be incorporated is disclosed in U.S. patent application Ser. No. 18/307,592, entitled “Meat Identification System and Method,” which was filed on Apr. 26, 2023 and is hereby incorporated herein by reference in its entirety.

Similar products may use a same type of bag as it enters the vacuum sealer. Furthermore, different types of products may also use a same type of bag, while other products may use different bags. This association between bags and products may be stored in data store 226. Analysis module 226 may determine, if a trend indicates that an abnormally high number of products are improperly vacuum sealed (e.g., exceeds a threshold percentage difference from historical values), whether those products are a same product or different products that would utilize a same bag. If it is only same products or different products that utilize the same bag that cause the trend to be indicative of a production error, analysis module 222 may determine that the production error may be a package quality error rather than any error with the machinery or user processes.

In some instances, communication module 220 may output an indication of the production error, including in the form of a visual, audible, or tactile alert.

In some instances, in response to determining that quality score is below a seal score threshold (e.g., the vacuum seal is likely to be improper), communication module 220 may perform a corrective action. In performing the corrective action, communication module 220 may output an alert (e.g., visual, audible, or tactile) notifying a user of the leak in the packaged product, or communication module 220 may control a sorting mechanism to remove the packaged product from a conveyor carrying the packaged product. In other instances, in response to determining that the quality score is above a seal score threshold (e.g., the vacuum seal is likely to be proper), communication module 220 may control a sorting mechanism to keep the packaged product on a conveyor carrying the packaged product. In still other instances, in response to determining that the quality score is above a first seal score threshold but below a second seal score threshold (e.g., it is not easily discernable whether the vacuum seal is proper or improper), communication module 220 may output an alert (e.g., visual, audible, or tactile) for a user to manually inspect the packaged product.

In some instances, analysis module 222 may also estimate, based on the one or more images, an amount of air inside packaging of the packaged product. In such instances, analysis module 222 may determine the quality score for the packaged product based at least in part on the one or more characteristics of the light and the estimated amount of air.

FIGS. 3A-6G are different perspective views of a lighting system and a camera system that are integrated into a product processing and packing system, in accordance with one or more techniques of this disclosure. The defect detection system that includes the lighting system and the camera system may be integrated into an automatic pack-off system. Below is one example of the defect detection system described herein, although other examples of the system are feasible and contemplated.

FIGS. 3A-3B are different perspective views of a lighting system and a camera system that are integrated into a product processing and packing system, in accordance with one or more techniques of this disclosure. Specifically, FIG. 3A is a perspective view of the lighting system and the camera in camera system enclosure 324 with front panel 323 installed via fasteners 332, while FIG. 3B is a perspective view of the lighting system and the camera in camera system enclosure 324 without the front panel installed.

In FIGS. 3A-3B, product processing system 10 includes classification system hood 320 and rig frame 322, which, in some examples, includes computers and electronics capable of performing at least some of the techniques described herein, such as computing device 210. In other examples, the computers and electronics capable of performing at least some of the techniques described herein, such as computing device 210, may be located in other places, such as camera system enclosure 324, HMI screen 334, or a separate device in wired or wireless communication with product processing system 10. In some instances, classification system hood 320 may do additional functions, such as classifying meat product 330 based on an analysis of a type of meat included within meat product 330 and directing meat product 330 to a proper processing location.

Product processing system 10 further includes side panels 326 and conveyor belt 328. Each of side panels 326 and conveyor belt 328 may be substantially uniform in color, and may be either the same color as one another or different colors (e.g., blue, black, or side panels 326 may be blue and conveyor belt 328 may be black). Side panels 326 and conveyor belt 328 may be uniformly colored in colors not typically found in meat (e.g., colors other than red, brown, and white) so that the camera system and computing device 210 may efficiently discern between side panels 326, conveyor belt 328, and meat product 330.

Conveyor belt 328 may transport meat product 330 from blower 18 into product processing system 10 and, specifically, under camera system enclosure 324 such cameras within camera system enclosure 324. Examples of those cameras include those within camera enclosures 336A-336C as shown in FIG. 3B. Also shown in FIG. 3B are lights 338A-338B to illuminate an area beneath camera system enclosure 324 on conveyor belt 328, including meat product 330, to capture better images with cameras in camera enclosures 336A-338B

FIGS. 3A and 3B further include human machine interface (HMI) monitor 334. HMI monitor 334 is a screen that the operator may use to control and monitor the pack-off system and the leaker detection system. The computer vision meat classification system (i.e., classification system hood 320) is in the large hood at the rear of the figure. It sits over the gapping scale system that helps increase the size of the gap between the meat products for sorting. Separating the products also helps the leaker and classification systems as it results in only one product per image.

In some instances, a camera in one of camera enclosures 336A-336C may be used as a sensor to determine when a meat product passes underneath camera system enclosure 324. In other instances, other sensors (e.g., photo eyes) may be used to detect the presence of meat product 330. This may help trigger the system to know a product is present and also has a role in communication with the classification system.

While only lights 338A and 338B are shown in FIG. 3B, in some instances, there may be another set of lights attached to the removed front panel 323 which has been removed and that are not shown in FIG. 3B.

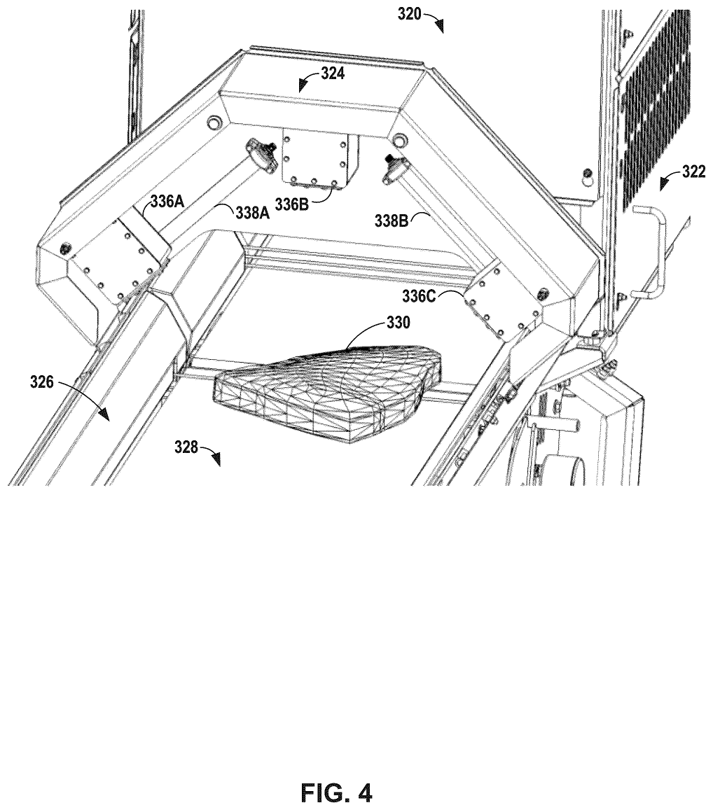

FIG. 4 is a perspective view of lights 338A and 338B and camera enclosures 336A-336C that are integrated into a product processing and packing system, in accordance with to one or more techniques of this disclosure. In some instances, a camera in one of camera enclosures 336A-336C may be used as a sensor to determine when a meat product passes underneath camera system enclosure 324. In other instances, other sensors (e.g., photo eyes) may be used to detect the presence of meat product 330. This may help trigger the system to know a product is present and also has a role in communication with the classification system.

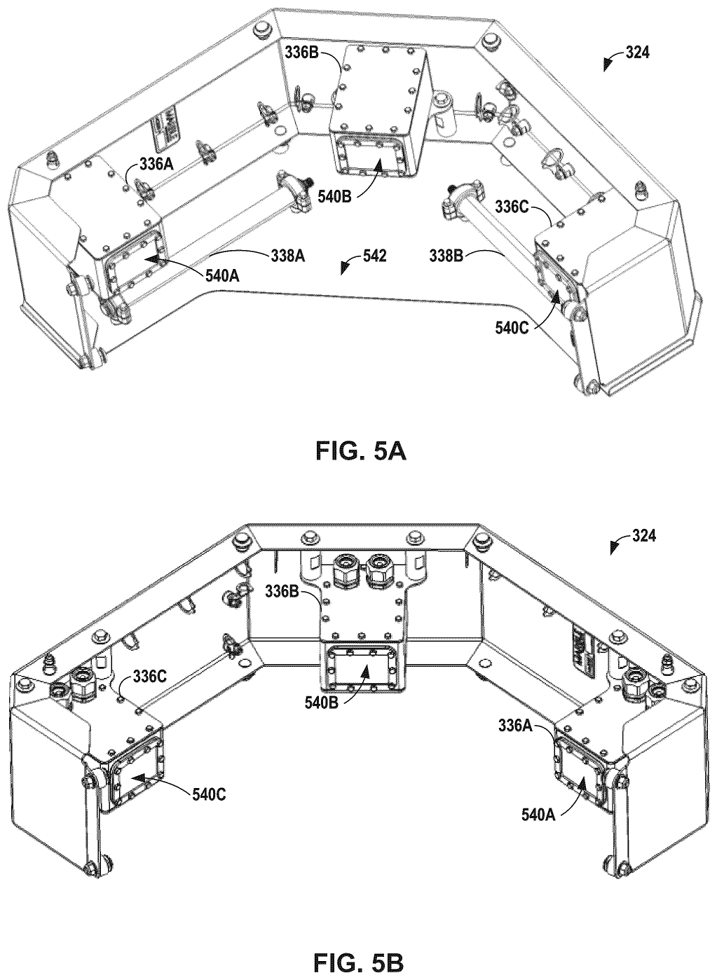

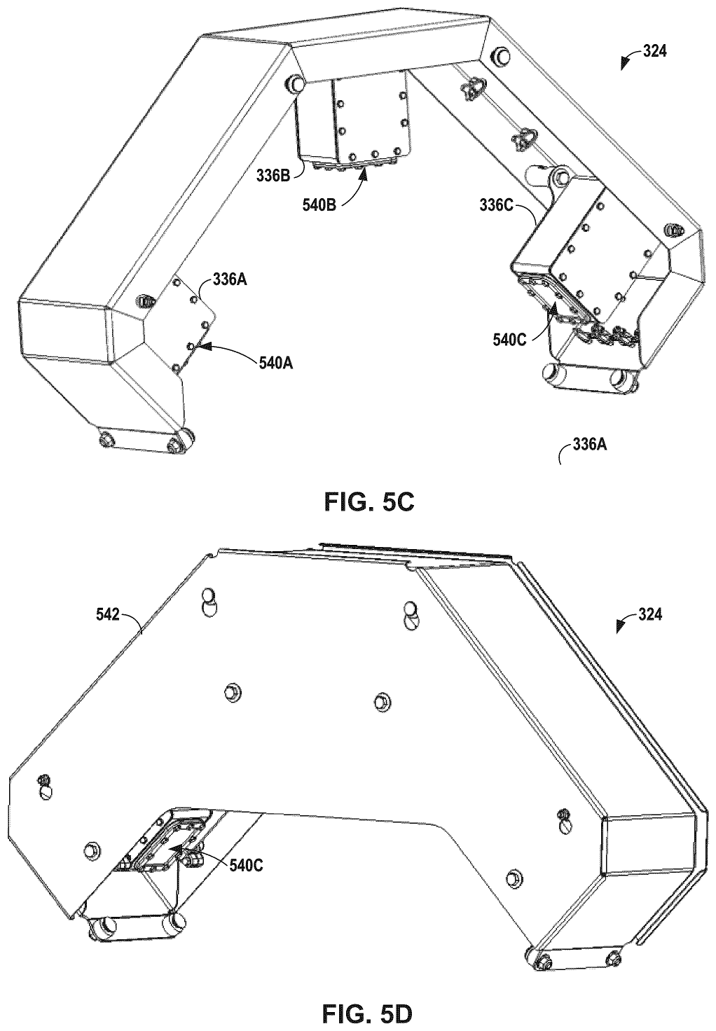

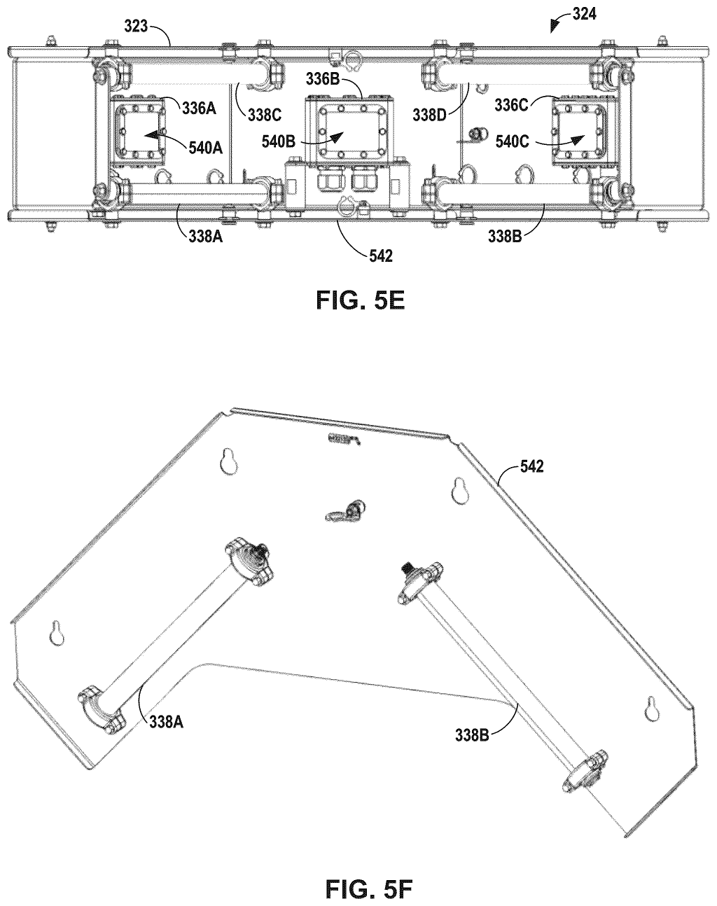

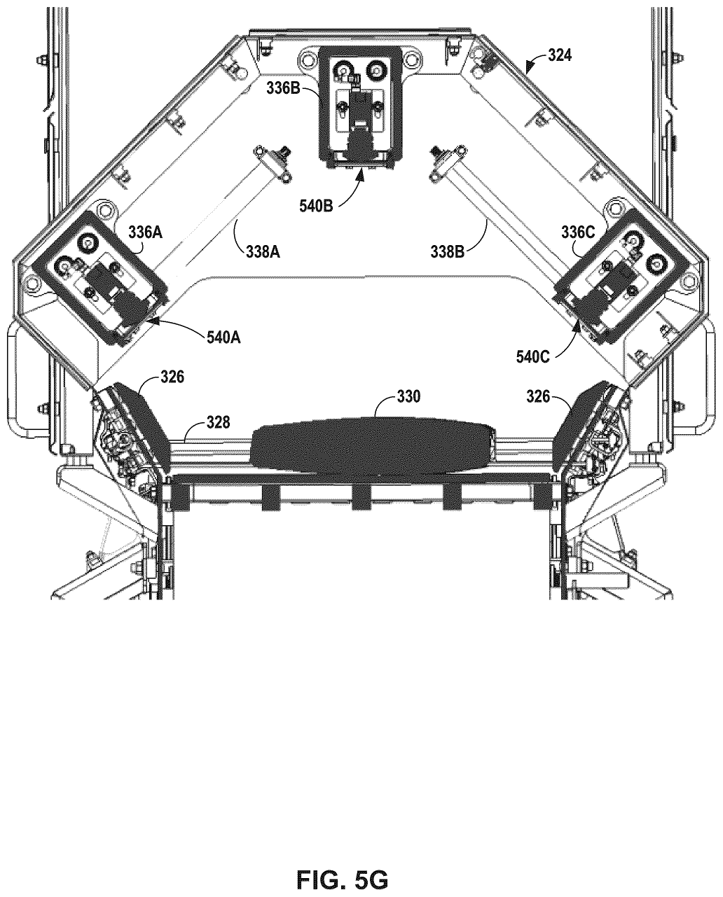

FIGS. 5A-5G are different perspective views of camera system enclosure 324, including lights 338A-338D and camera enclosures 336A-336C, which are integrated into a product processing system 10, in accordance with one or more techniques of this disclosure. Also visible in various of FIGS. 5A-5G are lenses of cameras 540A-540C within the respective camera enclosures 336A-336C, as well as back panel 542. In some instances, camera enclosure 336A-336C may have a clear panel on one end near lenses of cameras 540A-540C such that cameras 540A-540C may view objects through respective camera enclosures 336A-336C while still being completely enclosed to protect cameras 540A-540C from environmental factors.

FIG. 5A shows an example of camera system enclosure 324 with front panel 323 removed. Camera enclosures 336A-336C and cameras 540A-540C may be within a single welded arched frame. Pegs on the outside of the frame may be used as mounting points for the front panel. There may be two attachment points on each end of the arch that attach to the scale/gapper conveyor. Cable management rings are shown inside the arch which route all the cables between cameras 540A-540C and lights 338A-338D (lights 338C and 338D not shown).

FIG. 5B shows an example of camera system enclosure 324 with both front panel 323 and back panel 542, as well as lights 338A-338D, removed and the arch shape of camera system enclosure 324 flipped over to show the backside of camera enclosures 336A-336C, which contain the glands where the power and ethernet come from the cameras. Also shown is the hardware that may attaches cameras 540A-540C and camera enclosures 336A-336C to the frame of camera system enclosure 324.

FIG. 5C shows an example of another angle of the arch of camera system enclosure 324 with both front panel 323 and back panel 542 removed.

FIG. 5D shows an example of the arch of camera system enclosure 324 with back panel 542 included.

FIG. 5E shows an example of camera system enclosure 324 from the underside with front panel 323 and back panel 542 attached. In this view, lights 338A-338D are attached to the respective panels (i.e., lights 338A and 338B are attached to back panel 542, while lights 3338C and 338D are attached to front panel 323). When front panel 323 and back panel 542 are removed, the respective attached lights are also removed, allowing greater access to cameras 540A-540C and camera enclosures 336A-336C. The design also allows lights 338A-338D to be close in proximity to cameras 540A-540C but keeps lights 338A-338D themselves out of the field of view of cameras 540A-540C. Lights 338A-338D may create a harsh lighting environment to accentuate ripples and texture, which may be features of leakers.

FIG. 5F shows an example of back panel 542 with lights 338A and 338B mounted onto it.

FIG. 5G shows a section view through the center of product processing system 10, including camera system enclosure 324. FIG. 5G depicts an example field of view for each of cameras 540A-540C. The field of views may be set so each camera of cameras 540A-540C can see meat product 330 no matter where it sits on conveyor belt 328 (centered, to the right edge, to the left edge, etc.). The top camera (i.e., camera 540B) has the full field of view of conveyor belt 328 plus side panels 326.

Side panels 326 may be tapered out to allow most of the side of meat product 330 to be visible to one of cameras 540A-540C even when meat product 330 is far left or far right. In some instances, leaker features may be mostly or only visible on the side of meat product 330.

FIG. 5G also shows how the arch of camera system enclosure 524 may be bolted on to the flared sides of conveyor belt 328 with spacers.

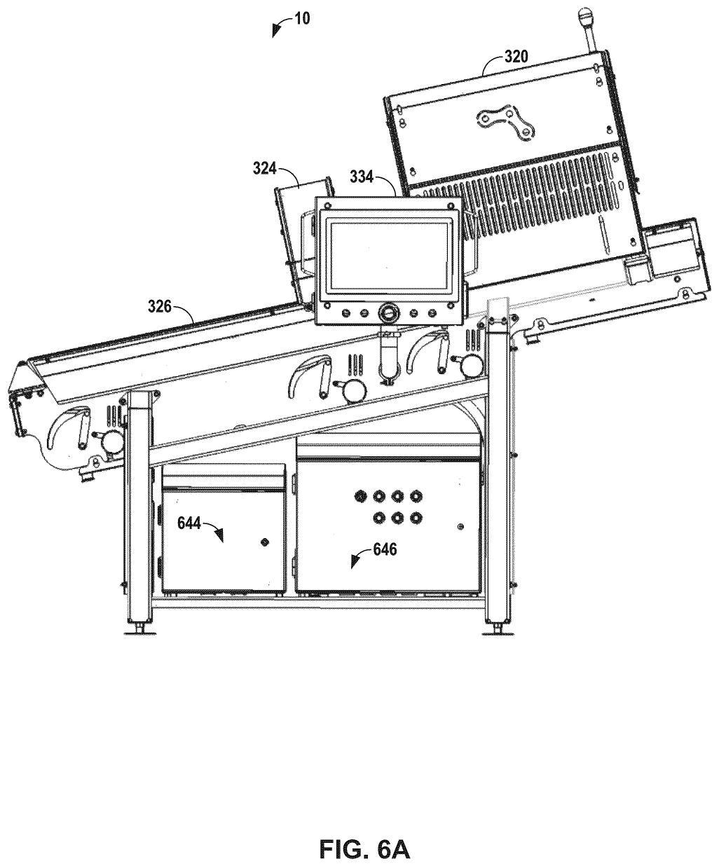

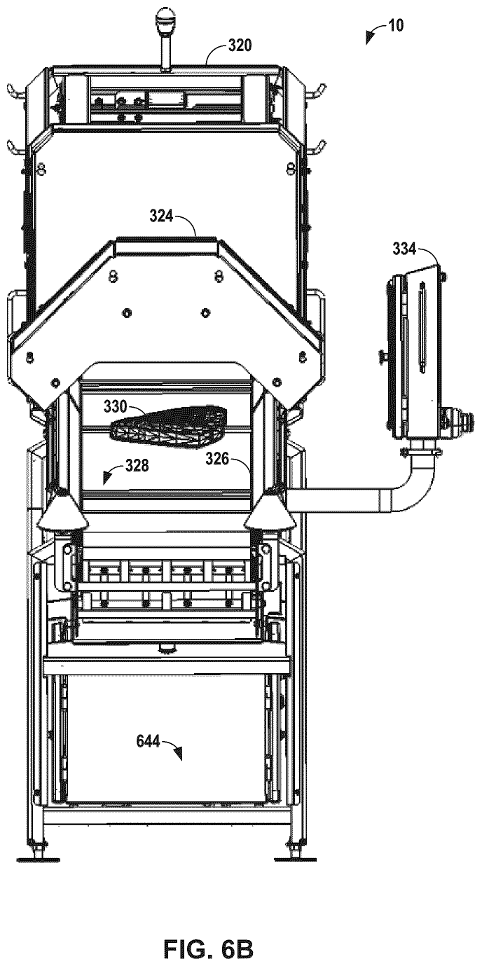

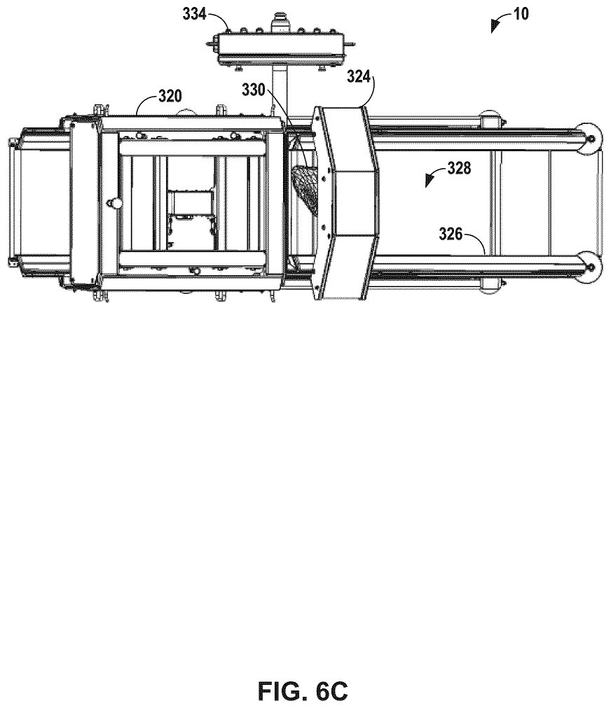

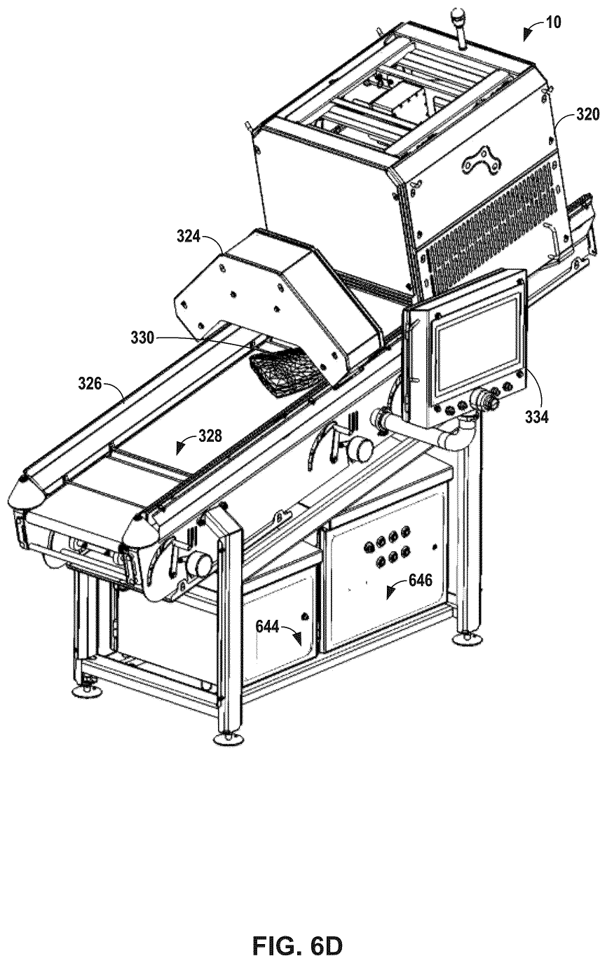

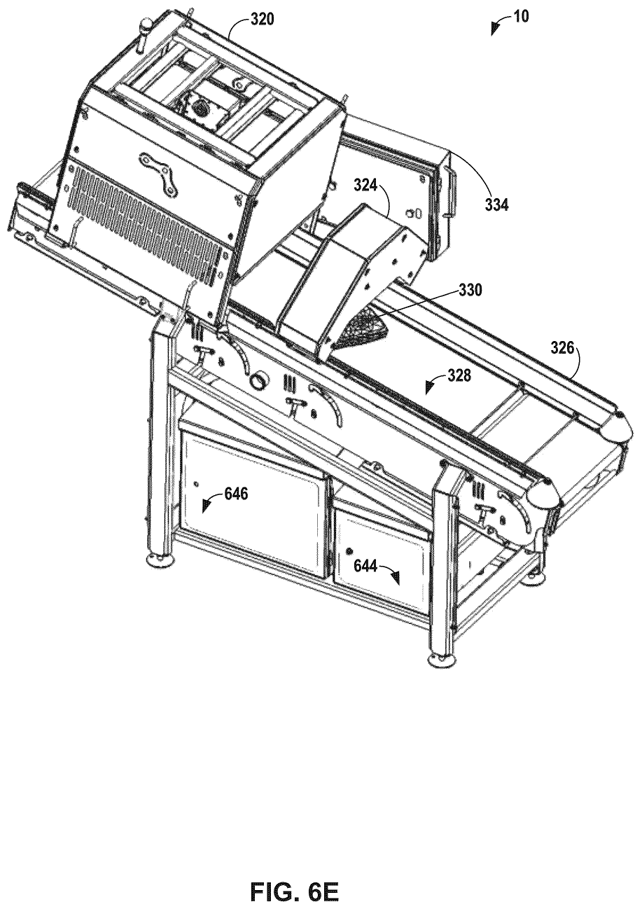

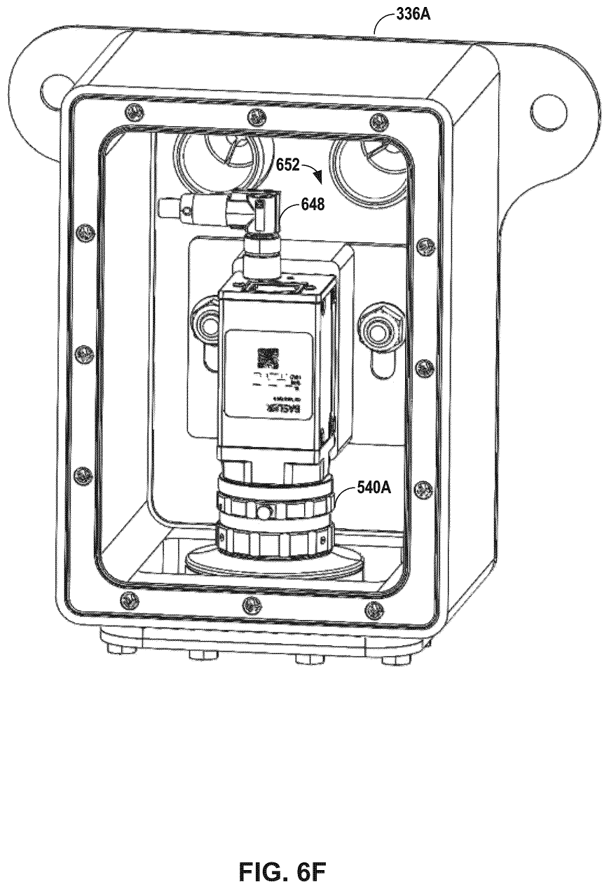

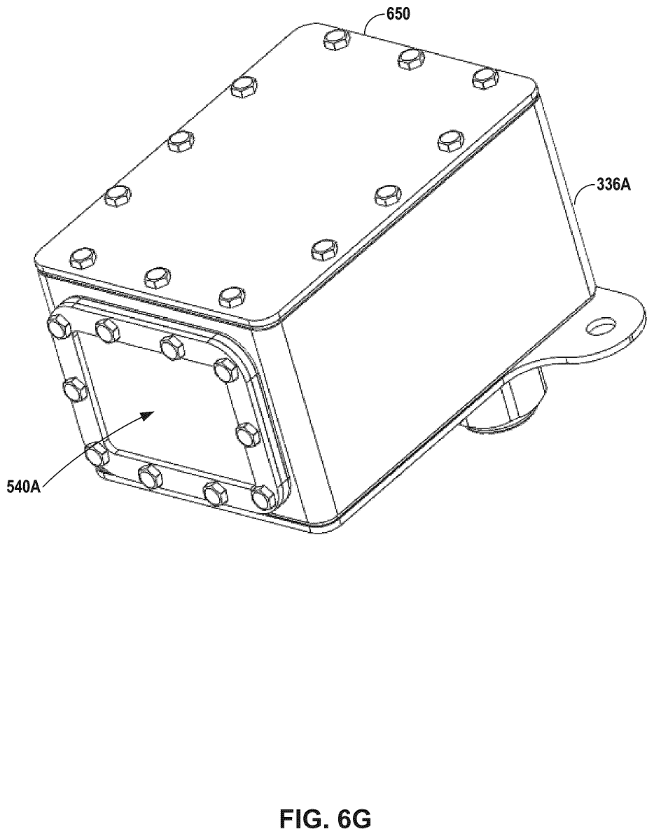

FIGS. 6A-6G are different perspective views of product processing system 10 that includes a lighting system and a camera system in camera system enclosure 324, in accordance with one or more techniques of this disclosure.

FIG. 6A shows an example side view of product processing system 10. As shown in FIG. 6A, product processing system 10 may include control panels 644 and 646, shown under side panel 326, HMI screen 334, and camera system enclosure 324. FIG. 6A also shows classification system hood 320, which is partially visible behind HMI screen 334.

FIG. 6B is an example infeed view showing meat product 330 entering product processing system 10 and camera system enclosure 324 via conveyor belt 328.

FIG. 6C is an example top down view of product processing system 10. Camera system enclosure 324, conveyor belt 328, side panels 326, classification system hood 320, and HMI screen 334 are all visible in FIG. 6C. In some instances, a minimum distance between classification system hood 320 and camera system enclosure 324 may be maintained so that there is no contamination of specific light settings (e.g., soft, diffuse light) in classification system hood 320 by the bright harsh light from camera system enclosure 324. Protecting against light contamination is also a reason for the shape of the front and back cover panels of camera system enclosure 324.

FIGS. 6D and 6E are examples of product processing system 10 from other angles.

FIG. 6F and FIG. 6G show example detailed views of the camera enclosures (e.g., camera enclosure 336A) within camera system enclosure 324.

Camera enclosure 336A has a back panel 652. There may be a unibody plastic enclosure that has set screws within the frame that are used to screw it together with a gasket in between. There may be dual ridges machined into the plastic around the enclosure to help create a tight, dual seal on camera enclosure 336A. There may be a back plate and a front plate. The back panel 652 is where the cable glands go through for cables going in and out and the mounting attachments. On the inside of the enclosure, there may be an aluminum plate block that is used to mount camera 540A and as a heat sink. It may be attached to back panel 652 which also acts as a heat sink to dissipate heat out of camera 540A. In some instances, camera 540A may be an industrial IP camera or an ethernet-based CV camera. Power cable 648 may plug into camera 540A. In some cases, camera 540A may have a fixed aperture lens or a wide angle lens. In some instances, camera 540A may have focus rings on it. Camera 540A may be brought in and slid down so that the lens is almost touching the enclosure cover to ensure minimal reflection back.

The bottom may have similar holes and mounting as the front gasket. It may have a scratch-resistant polycarbonate gasket, a stainless ring around it, and the whole stack bolts in.

Camera enclosure 336A may be made without front cover 650, such as a solid block of plastic, but it would be more difficult to work with as a technician would have to take it apart to reach camera 540A. Front cover 650 may give better access to these internal components. Front cover 650 may be unscrewed and removed without altering the alignments of camera 540A.

FIGS. 6A-6G are different perspective views of a product processing and packing system that includes a lighting system and a camera system, in accordance with one or more techniques of this disclosure. The leaker detection system described herein may be incorporated into an automated pack-off system. However, the detection system could be installed separately. If installed in isolation, the leaker detection system would largely be a data collection and analysis tool. A facility could track the number of leakers generated and use the data to identify trends (e.g., more leakers today from a specific vacuum sealer). In some examples, the leaker detection system could be connected to a simple sortation system that redirects leakers but does not provide product chutes and boxing stations.

The techniques of this disclosure may identify defective packages on production lines at production speeds without disrupting processing operations. Its small size and inline nature are critical advantages over offline, slow, tedious methods.

Throughout this disclosure, reference is made to products that have been vacuum sealed, wherein the defect detection system analyzes the packaged product to see if the product was vacuum sealed. While these techniques are described using vacuum sealed products, these techniques could be utilized for any product that is packaged to be in any low oxygen state. With regard to meat, storing the meat in this low oxygen state is necessary to reduce spoilage, other damage to the meat, and to wet age for the meat for food-grade quality.

The defect detection system described herein may include an image data capture rig and a computer vision system that classifies meat products as leaker (defective) or non-leaker (not defective). For the purposes of this disclosure, a packaged product that is properly vacuum sealed may be referred to as a non-leaker while a packaged product that is not properly vacuum sealed may be referred to as a leaker, even if the detail causing the package to be improperly vacuum sealed is not the seal in and of itself. For instance, a defective product that is not properly vacuum sealed may have a seal that is intact but may have extraneous air on an interior of the package. There may not be a literal leak in the package, but the extraneous air may lead to a classification as a “leaker” due to the improper vacuum seal.

The image data capture rig may include a metal frame that provides structure to the rig. The rig is positioned above a conveyor that conveys meat products from a vacuum sealer under the image data capture rig. The frame has bracket rods that attach the frame to the product classification system of the automated pack-off system. In alternate examples, the bracket rods of the image data capture rig frame may attach to supports extending above the conveyor to hold the image data capture rig in position above the conveyor when it is not placed adjacent to the product classification system. In still other examples, the image data capture rig may integrate with a classification hood structure.

The conveyor may have an angled panel on each side. The side panel may be made of plastic material the same color as the conveyor belt. The purpose of this panel is to provide a single color background in the images of meat products collected by the three cameras of the image data capture rig.

Attached to the image data capture rig frame are a number of camera enclosures, such as three camera enclosures. Note that in some examples, fewer or a greater number of cameras and camera enclosures may be present. Each camera enclosure contains a Basler camera (Ace 2 basic GigE). In other embodiments, a different camera may be included, such as an Intel RealSense camera. Each Basler camera is equipped with a lens. The lens may be chosen to provide a desired field of view, the desired depth of field, and may not have adjustable components that may loosen over time. In other examples, different lenses could be chosen.

As shown in FIGS. 3A-6G, the three cameras may be arranged in a triangular manner in the center of the image data capture rig. The field of view of the central camera covers the width of the conveyor and captures an image of the entire top of the meat products passing along the conveyor under the image data capture rig. The side cameras are angled to capture an image of the side of the meat products. The field of view of both angled cameras covers the entire conveyor. Meat products, typically, are randomly and chaotically discharged onto the conveyor by the vacuum sealer. Some products are positioned in the center of the conveyor. Other products are shifted to the right or left edge of the conveyor. The position of the side cameras and the selected camera field of view, to capture images of the side of the meat product at as many positions (right center left) of meat products along the conveyor as possible.

Each camera enclosure may include a CCD camera and lens that looks through a window at the products. The enclosure may be made of food-grade material like Acetal plastic, silicon rubber, and stainless steel that can withstand the harsh chemicals used to clean food processing equipment and can withstand high-pressure hot water cleaning. There are internal mounting brackets that both position the camera but also act as a heatsink to keep the camera within its operating temperature. Some examples may include a single unibody enclosure that holds all cameras to minimize cable entry points.

The image data capture rig may have light covers, such as four L-shaped stainless light covers. The light covers may be mounted onto the image data capture rig frame in a tent or triangular position. The light covers have cutouts for the side cameras. Made of food-grade stainless steel, the covers direct light down onto the conveyor and keep the light from shining in the eyes of the human operators working near the rig. In other examples, rather than including light covers, the lights may be attached to removable panels, with the panels providing the rigid attachment structure to house the lights and protect the lights both from shining into the eyes of human operators and from environmental factors around the system.

On the underside of the light covers are brackets for holding the image data capture rig lights. The system has 4 lights (in some embodiments, the number and position of the lighting could be altered). One light sits in front of the center axis of the rig along which the camera enclosures are mounted and one sits behind this center axis on both the right and left arms of the image data capture rig. The position was chosen to create harsh lighting conditions within the three cameras' fields of view, although other positions and angles are possible that still allow for similar techniques. The harsh lighting glares off the plastic packaging of the meat products creating glare patterns that differ between leaker and non-leaker products.

In alternate examples, the lights are replaced with laser lighting (point lasers, line lasers, grid lasers, etc). The scatter of the laser lighting as it reflects off the plastic packaging is captured in the image data. The diffusion of laser light in the air packet may create a heat map of light dispersion. In such examples, the computing device may input the heat map through a neural network for analysis to analyze diffusion patterns of the laser light as compared to natural variance of a properly vacuum sealed product.

Images of meat products are collected by the image data capture rig. In some examples, for long products, the full product may not fit into the field of view of the cameras. In this case, multiple frames of each product may be collected and stitched together to show the full product. Each frame consists of three images, one from each of the three cameras at the different angles. The Deep Learning CNN model may evaluate the stitched images for signs of leaking. This allows the model to either evaluate each frame by itself or stitched together and evaluate the product as a whole. The model also detects the leading and trailing edges of the product to track its progress on the conveyor. Before the leaker detention rig there is a set of gapping conveyors to ensure products are in a single row as they pass under the leaker detection rig.

The Leaker Detection Computer Vision Model utilized for the analysis herein (e.g., analysis module 222 of FIG. 2) may be a deep learning model that consists of a convolutional neural network (CNN) encoder that takes as input an image frame captured from the three cameras within the leaker detection system. The images of each camera are passed through the encoder concurrently and the encoded feature vectors of the three image inputs are concatenated and sent to a classifier head that classifies the product in the images as either leaker or non-leaker. Using the leaker detection model, the characteristic glare patterns exhibited by leaked products can be observed by the model with a pre-specified lightning environment and used to distinguish between leaked and non-leaked products. Since the camera fields-of-view (FOV) are small along the conveyor belt direction (e.g., ˜8 inches at the conveyor belt), the algorithm may make multiple classifications on a product as it passes through the scan area, where the model is observing a different portion of the product at each instance. The majority vote of the classifications for a product is taken as the final classification for that product. The supervisory software may then bypass products classified as “leaker” from the sortation chutes.

The lights in the image data capture rig create a harsh lighting environment within the cameras' field of view. The harsh light glares off the shiny plastic packaging. Air bubbles or plastic wrinkles are common on defectively sealed plastic packages. In addition, the clear plastic package may appear “hazy” on leakers due to the presence of air between the plastic and meat products. The harsh light reflects differently off the features seen in the defective plastic packaging creating differences in the glare pattern of the product in an image. Deep learning CNNs are well suited to detecting such visual patterns.

The model's classification of leaker or non-leaker may be integrated into a pack-off system's supervisory software so that products identified as leakers are not sorted to boxing stations but rather routed to an alternate area for repackaging.

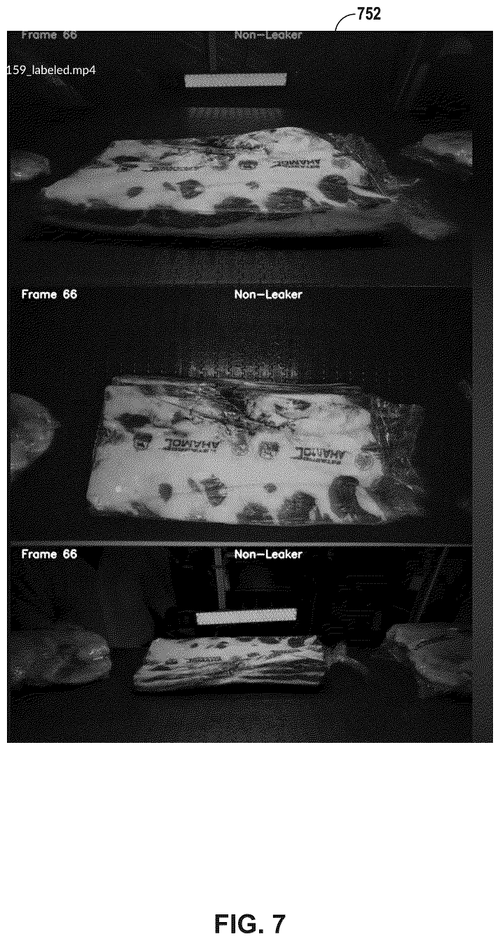

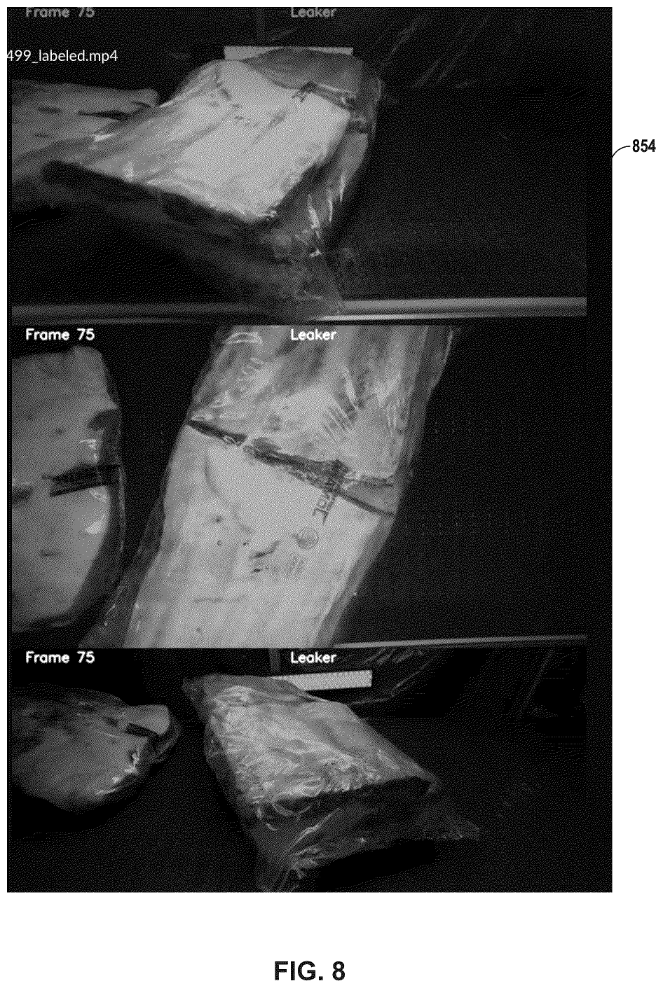

FIG. 7 is an example set of images 752 of packaged products without detected leaks, in accordance with one or more techniques of this disclosure. FIG. 8 is an example set of images 854 of packaged products with detected leaks, in accordance with one or more techniques of this disclosure. As shown in FIG. 7, a packaged product that has been properly sealed will have minimal glare given the tight fit of the plastic to the product itself. Meanwhile, as shown in FIG. 8, the reflection of the light on a packaged product that has not been properly sealed will have a harsher glare and a greater number of wrinkles will be present over the product itself rather than just at the seams.

Pairs of LED light bars sandwich the side cameras, imparting very hard, high-intensity white light. When a product is well sealed (non-leaked) the glare in this environment gives the product a glossy wet look, as shown in FIG. 7. Whereas if it is leaked, the wrinkles, tight bubbles, and gaps between the meat and plastic are better illuminated and more readily apparent in the image data in this harsh lighting environment, as shown in FIG. 8.

In some instances, the cameras may capture image data at every timestamp as products move through the leaker detection system to capture multiple instances per product. Instances may be individual images or sets of images (e.g., sets of three images) from the set of cameras. Each instance is fed into a deep learning model trained on data to detect when a product is leaked or not. In non-leaked products, the camera vision system may detect minimal or no glare patterns, as well as details of the product like fat and lean meat. As shown in FIG. 8, the images include other glare patterns, saturation of glare, and haziness. The deep learning model has been trained on tens of thousands of meat products (leaked and non-leaked) and has learned these feature patterns. For each instance (set of images), the computing device may run an inference through the model. The computing device may then perform an aggregation method that comes up with the final classification.

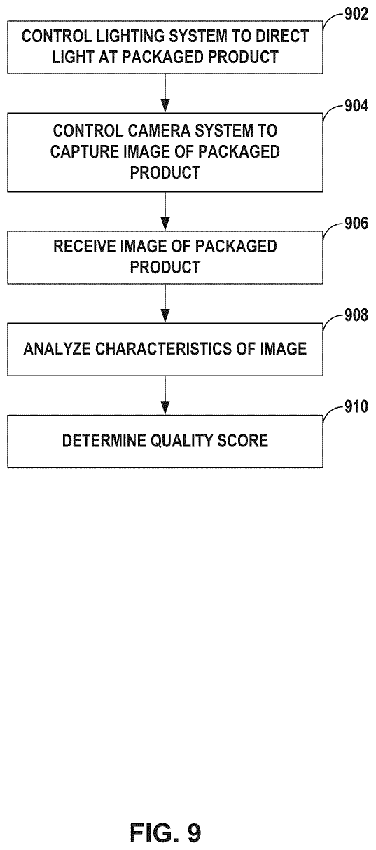

FIG. 9 is a flow diagram illustrating an example method for evaluating a vacuum seal on a packaged product, in accordance with one or more techniques of this disclosure. The techniques of FIG. 9 may be performed by one or more processors of a computing device, such as system 10 of FIG. 1 and/or computing device 210 illustrated in FIG. 2. For purposes of illustration only, the techniques of FIG. 9 are described within the context of computing device 210 of FIG. 2, although computing devices having configurations different than that of computing device 210 may perform the techniques of FIG. 9.

In accordance with the techniques of this disclosure, communication module 220 controls a lighting system to direct light at a packaged product (902). Communication module 220 controls a camera system to capture one or more images of the packaged product while the lighting system is directing the light at the packaged product (904). Communication module 220 receives the one or more images of the packaged product captured while the lighting system is directing light at the packaged product (906). Analysis module 222 analyzes one or more characteristics of the images (e.g., one or more characteristics of light in the one or more images) (908). Analysis module 222 determines, based on the one or more characteristics in the one or more images, a quality score indicating whether the packaged product was properly vacuum sealed.

Although the various examples have been described with reference to preferred implementations, persons skilled in the art will recognize that changes may be made in form and detail without departing from the spirit and scope thereof.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structures or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Various examples of the disclosure have been described. Any combination of the described systems, operations, or functions is contemplated. These and other examples are within the scope of the following claims.

1. A method comprising:
controlling, by one or more processors, a lighting system to direct light at a packaged product;
controlling, by the one or more processors, a camera system to capture one or more images of the packaged product while the lighting system is directing the light at the packaged product;
receiving, by the one or more processors, the one or more images of the packaged product captured while the lighting system is directing light at the packaged product;
analyzing, by the one or more processors, one or more characteristics of the light in the one or more images; and
determining, by the one or more processors and based on the one or more characteristics of the light in the one or more images, a quality score indicating whether the packaged product was properly vacuum sealed.

2. The method of claim 1, wherein analyzing the one or more characteristics of the light comprises one or more of:
analyzing one or more glare patterns of the light reflecting off the packaged product, and
analyzing one or more scatter signatures of the light on the packaged product.

3. The method of claim 2, wherein the one or more scatter signatures comprise manifestations of green wavelength spectrum light shined into the packaged product.

4. The method of claim 3, wherein the lighting system comprises one or more lasers that emit green wavelength spectrum light.

5. The method of claim 1, wherein the packaged product comprises a vacuum sealed food product.

6. The method of claim 1, wherein the camera system comprises a plurality of camera devices.

7. The method of claim 6, wherein the camera system further comprises a camera enclosure surrounding each respective camera of the plurality of cameras.

8. The method of claim 6, wherein a first camera of the plurality of cameras is positioned above a conveyor carrying the packaged product, wherein a second camera of the plurality of cameras is positioned on a first side of the conveyor, and wherein a third camera of the plurality of cameras is positioned on a second side of the conveyor.

9. The method of claim 1, wherein the packaged product comprises a first packaged product in a plurality of packaged products being carried by a conveyor, and wherein the method further comprises:
controlling, by the one or more processors, a set of gapping conveyors to move the plurality of packaged products into a single row prior to passing the lighting system and the camera system.

10. The method of claim 1, wherein analyzing the one or more characteristics of the light in the one or more images comprises:
for each of the one or more images:
inputting, by the one or more processors, the respective image into a model trained with previous images of packaged products that contain leaks and with previous images of packaged products that do not contain leaks; and
comparing, by the one or more processors and using the model, the one or more characteristics of the light in the respective image to one or more characteristics of the light for the model; and
determining, by the one or more processors, the quality score based on each of the comparisons for each of the one or more images.

11. The method of claim 1, wherein each of the one or more images comprise one or more of:
an image captured by a same camera device at a unique time to show a different portion of the packaged product, and
an image captured by a different camera device to show a different angle of the packaged product.

12. The method of claim 1, further comprising:
in response to determining that quality score is below a seal score threshold, performing, by the one or more processors, a corrective action.

13. The method of claim 12, wherein performing the corrective action comprises one or more of:
outputting, by the one or more processors, an alert notifying a user of the leak in the packaged product, and
controlling, by the one or more processors, a sorting mechanism to remove the packaged product from a conveyor carrying the packaged product.

14. The method of claim 1, further comprising:
in response to determining that the quality score is above a seal score threshold, controlling, by the one or more processors, a sorting mechanism to keep the packaged product on a conveyor carrying the packaged product.

15. The method of claim 1, further comprising:
in response to determining that the quality score is above a first seal score threshold but below a second seal score threshold, outputting, by the one or more processors, an alert for a user to manually inspect the packaged product.

16. The method of claim 1, wherein the one or more characteristics of the light comprise one or more of:
a glare pattern created by one or more of air bubbles,
plastic wrinkles in the packaged product,
haze on a plastic exterior of the packaged product,
a color contrast between packaging of the packaged product and a product inside the packaging,
blood or other liquid in or around a seal of the packaged product,
wrinkles around the seal of the packaged product,
contamination on one or both sides of the seal of the packaged product,
a burn through of the seal of the packaged product, and
a sign of the seal of the packaged product lacking integrity.

17. The method of claim 1, wherein the lighting system comprises one or more of LED lights or fluorescent lights.

18. The method of claim 1, wherein the quality score comprises one or more of:
a probability of the packaged product being properly vacuum sealed,
a probability of the packaged product being improperly vacuum sealed, and
a quantitative value based on a comparison of the one or more images to one or more images of a machine learning model.

19. The method claim 1, wherein the packaged product comprises a first packaged product of a plurality of packaged products, and wherein the method further comprises:
for each of the plurality of packaged products:
receiving, by the one or more processors, the one or more images of the respective packaged product captured while the lighting system is directing light at the respective packaged product;
analyzing, by the one or more processors, one or more characteristics of the light in the one or more images of the respective packaged product; and
determining, by the one or more processors and based on the one or more characteristics of the light in the one or more images of the respective packaged product, a quality score for the respective packaged product indicating whether the respective packaged product was properly vacuum sealed.

20. The method of claim 19, further comprising:
determining, by the one or more processors and based on each of the quality scores for the packaged products of the plurality of packaged products, trend data for the plurality of packaged products.

21. The method of claim 19, further comprising:
determining, by the one or more processors and based on the quality scores for the plurality of packaged products, a failure rate for the plurality of packaged products;
comparing, by the one or more processors, the failure rate to a historical failure rate;
in response to the failure rate exceeding the historical failure rate by a threshold amount, determining, by the one or more processors, that a production error is present.

22. The method of claim 21, wherein the production error comprises one or more of a mechanical error, a user error, or a package quality error.

23. The method of claim 21, further comprising:
outputting, by the one or more processors, an indication of the production error.

24. The method of claim 1, further comprising:
estimating, by the one or more processors and based on the one or more images, an amount of air inside packaging of the packaged product; and
determining, by the one or more processors, the quality score for the packaged product based at least in part on the one or more characteristics of the light and the estimated amount of air.

25. A packing system comprising:
a lighting system;
a camera system; and
one or more processors configured to:
control the lighting system to direct light at a packaged product;
control the camera system to capture one or more images of the packaged product while the lighting system is directing the light at the packaged product;
receive the one or more images of the packaged product captured while the lighting system is directing light at the packaged product;
analyze one or more characteristics of the light in the one or more images; and
determine, based on the one or more characteristics of the light in the one or more images, a quality score indicating whether the packaged product was properly vacuum sealed.

26. The packing system of any one or more of claim 25, wherein the system further comprises a conveyor that moves the packaged product throughout the packing system.

27. The packing system of claim 26, further comprising a first angled panel on a first side of the conveyor and a second angled panel on a second side of the conveyor, wherein each of the first angled panel and the second angled panel are a same color as the conveyor such that the one or more images include the packaged product, the light directed at the packaged product, and a monotone background.

28. A non-transitory computer-readable storage medium having stored thereon instructions that, when executed, cause one or more processors of a computing device to:
control a lighting system to direct light at a packaged product;
control a camera system to capture one or more images of the packaged product while the lighting system is directing the light at the packaged product;
receive the one or more images of the packaged product captured while the lighting system is directing light at the packaged product;
analyze one or more characteristics of the light in the one or more images; and
determine, based on the one or more characteristics of the light in the one or more images, a quality score indicating whether the packaged product was properly vacuum sealed.
